(19) United States
(12) Patent Application Publication
(10) Pub. No.: US 2012/0079618 A1
(43) Pub. Date: Mar. 29, 2012

(54) BITTER MELON VARIETY CBM12 AND PRODUCTS THEREFROM

(75) Inventors: Chittaranjan Kole, Clemson, SC (US); Phullara Kole, Clemson, SC (US); Bode A. Olukolu, Raleigh, NC (US); Albert G. Abbott, Clemson, SC (US)

(73) Assignee: Clemson University

(21) Appl. No.: 13/179,952

(22) Filed: Jul. 11, 2011

Related U.S. Application Data
(60) Provisional application No. 61/364,938, filed on Jul. 16, 2010.

(51) Int. Cl. A01H 1/02; A01H 5/00; A01H 5/08; C12N 5/04; A01H 5/10

(52) U.S. Cl. 800/260; 435/410; 800/309

(57) ABSTRACT

The present invention relates to bitter melon breeding and products, in particular, to a new bitter melon variety designated CBM12.

### STATEMENT OF PRIORITY

This application claims the benefit, under 35 U.S.C. §119 (e), of U.S. Provisional Application Ser. No. 61/364,938, filed Jul. 16, 2010, the entire contents of which are incorporated by reference herein.

### STATEMENT OF GOVERNMENT SUPPORT

Aspects of this invention were supported by funding under Grant No. 2009-35301-15041 from the USDA National Institute of Food and Agriculture. The U.S. Government has certain rights in this invention.

### FIELD OF THE INVENTION

The present invention relates to bitter melon breeding and products, in particular, to a new bitter melon variety designated CBM12.

### BACKGROUND OF THE INVENTION

Bitter melon (Momordica charantia L, Family: Cucurbitaceae) is a “functional food” that can be consumed as a vegetable and as a medicine. This plant contains an array of therapeutic chemicals active against many diseases, particularly cancer and diabetes. For example, cucurbitacin-B, lycopene, beta-carotene, and alpha- and beta-momorcharin are active against cancer while charantin and polypeptide-p, also known as plant insulin, possess antidiabetic properties. However, the popularly grown varieties have low contents of these bioactive compounds, albeit possessing desirable fruit quality traits. In contrast, some genotypes of the wild botanical variety M. charantia var. muricata are rich in these phytomedicines. Therefore, it would be worthwhile to introgress the genes controlling bioactives from muricata into the popular varieties belonging to the botanical variety charantia to develop dual purpose hybrids.

There are numerous stages in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The aim is to combine in a single variety an improved combination of desirable traits from the parental germplasm. These important traits may include higher yield, resistance to diseases and insects, better stems and roots, tolerance to drought and heat, improved nutritional/medicinal quality, and better agronomic characteristics.

Choice of breeding methods depends on the mode of plant reproduction, the heritability of the trait(s) being improved, and the type of variety used commercially (e.g., F₁ hybrid variety, pure line variety, etc.). For highly heritable traits, a choice of superior individual plants evaluated at a single location may be effective, whereas for traits with low heritability, selection can be based on mean values obtained from replicated evaluations of families of related plants. Popular selection methods commonly include pedigree selection, modified pedigree selection, mass selection, and recurrent selection.

The complexity of inheritance influences the choice of breeding method. Backcross breeding is used to transfer one or a few favorable genes for a highly heritable trait into a desirable variety. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program generally includes a periodic, objective evaluation of the efficiency of the breeding procedure. Evaluation criteria vary depending on the goals and objectives, but should include gain from selection per year based on comparisons to an appropriate standard, overall value of the advanced breeding lines, and number of successful varieties produced per unit of input (e.g., per year, per dollar expended, etc.).

The development of new bitter melon varieties involves the development and selection of bitter melon breeding lines, the crossing of these breeding lines and selection of superior hybrid crosses. Hybrid combinations are identified and developed on the basis of certain gene traits such as leaf size or color, flower color, disease resistance or herbicide resistance, phytomedicine content, and the like, which are expressed in a hybrid. Additional data, such as yield and quality traits, on parental lines as well as the phenotype of the hybrid influence the breeder's decision to continue with the specific hybrid cross.

Pedigree breeding and recurrent selection breeding methods are used to develop true breeding varieties from breeding populations. Breeding programs combine desirable traits from two or more varieties or various broad-based sources into breeding pools from which varieties are developed by selfing or alternatively, by creating doubled-haploids, and selection of desired phenotypes. The new varieties are evaluated to determine which have commercial potential.

Pedigree breeding is commonly used for the improvement of self-pollinating crops and parental lines for hybrids. Two parents which possess favorable, complementary traits are crossed to produce an F₁. An F₂ population is produced by selfing one or several F₁ plants. Selection of the best individuals may begin in the F₂ population; then, beginning in the F₃, the best individuals in the families are selected. Replicated testing of families can begin in the F₄ generation to improve the effectiveness of selection for traits with low heritability. At an advanced stage of inbreeding (i.e., F₆ and F₇), the best lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous variety or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent (e.g., variety) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂, individuals. The number of plants in a population may decline in each generation due to failure of some seeds to germinate or some plants to produce at least one seed. As a result, not all of the F₂ plants originally sampled in the population will be represented by a progeny when generation advance is completed.

In a multiple-seed procedure, breeders harvest seeds from one or more flowers from each plant in a population and pool them to form a bulk. Part of the bulk is used to plant the next generation and part is put in reserve. The procedure has been referred to as modified single-seed descent technique.

Proper testing should detect any major faults and establish the level of superiority or improvement over current varieties. In addition to showing superior performance, the breeder should consider whether there is a demand for a new variety that is compatible with industry standards or which creates a new market. The introduction of a new variety will incur additional costs to the seed producer, the grower, the processor and the consumer, for special advertising and marketing, altered seed and commercial production practices, and new product utilization. The testing preceding release of a new variety should take into consideration research and development costs as well as technical superiority of the final variety. For seed-propagated varieties, it must be feasible to produce seed easily and economically.

### BRIEF DESCRIPTION OF THE DRAWINGS

FIG. 1. Amplified fragment length polymorphism (AFLP) marker profile of 20 bitter melon varieties and two advanced breeding lines (CBM9 and CBM18). Lanes 1-22 indicate the genotypes as listed in Table 2. The lane 21 represents the fingerprint of CBM12.

FIG. 2. Fruit traits of CBM12 and close-up comparison to Taiwan White.

FIG. 3. Fruit traits of six randomly selected cultivars for comparison with CBM12 in FIG. 2.

FIG. 4. Relative content of cucurbitacin-B and charantin (in mg/g of powder from lyophilized fruit chips) of 18 varieties and one advanced breeding line (CBM18).

### SUMMARY OF THE INVENTION

The present invention relates to a new and distinctive bitter melon variety designated CBM12, having desirable agronomic and characteristics in combination with increased phytomedicine content.

Thus, some aspects of the present invention provide a bitter melon seed designated CBM12, representative seed of said CBM12 having been deposited under ATCC Accession No. ______.

Further provided herein is a bitter melon plant, or a part thereof, produced by the seed designated CBM12. Also provided herein is pollen of such plant as well as an ovule of such plant of this invention.

In further aspects, the present invention further provides a bitter melon plant, or a part thereof, having all the physiological and morphological characteristics of CBM12, said CBM12 having been deposited under ATCC Accession No. ______.

In additional aspects of this invention, tissue culture of regenerable cells of any of the plant, or part thereof, of this invention is provided. In such tissue culture, the regenerable cells can be from plant parts such as leaves, pollen, embryos, cotyledons, hypocotyls, roots, root tips, anthers, flowers and any part thereof, ovules, shoots, stems, stalks, pith and fruit (e.g., gourd, pepo), including embodiments wherein the regenerable cells are callus or protoplasts derived therefrom.

Further aspects of this invention include a bitter melon plant regenerated from the tissue culture of this invention and expressing all the morphological and physiological characteristics of CBM12, said CBM12 having been deposited under ATCC Accession No. ______.

Also provided herein is a method for producing a first generation (F₁) hybrid bitter melon seed comprising crossing a plant of this invention (CBM12) with a different bitter melon plant and harvesting the resultant first generation (F₁) hybrid bitter melon seed. An F₁ hybrid bitter melon seed produced by this method, as well as an F₁ hybrid plant, or a part thereof, grown from such seed is provided herein as well.

The present invention also provides a method for producing hybrid bitter melon seed comprising crossing two bitter melon plants and harvesting the resultant hybrid bitter melon seed, wherein at least one bitter melon plant is the bitter melon plant of this invention (CBM12).

In further aspects of this invention, a method is provided for producing a CBM12-derived bitter melon plant comprising: (a) crossing CBM12, representative seed of said CBM12 having been deposited under ATCC Accession No. ______, with a second bitter melon plant to yield progeny bitter melon seed; (b) growing said progeny bitter melon seed, under plant growth conditions, to yield said CBM12-derived bitter melon plant. Also provided herein is a CBM12-derived bitter melon plant, or a part thereof, produced by such method.

Additionally provided herein is the bitter melon plant, or a part thereof, of this invention, wherein the plant or a part thereof has been transformed so that its genetic material comprises one or more transgenes operably linked to one or more regulatory elements.

Also provided herein is a method for producing a bitter melon plant that contains in its genetic material one or more transgenes, comprising crossing the bitter melon plant of this invention described above with either a second plant of another bitter melon line, or a non-transformed bitter melon plant of this invention, wherein progeny are produced, so that the genetic material of the progeny that result from the cross comprises the transgene(s) operably linked to one or more regulatory elements. In such embodiments, the transgene can be, for example, a transgene the expression of which confers herbicide tolerance, insect resistance, disease resistance, nematode resistance, tolerance to abiotic stress, yield enhancement, improved fruit characteristics, altered reproductive capability, altered chemical composition and any combination thereof. The present invention further encompasses a bitter melon plant, or a part thereof, produced by this method.

In addition, the present invention provides a method for developing a bitter melon plant in a bitter melon plant breeding program using plant breeding techniques, which include employing a bitter melon plant, or a part thereof, as a source of plant breeding material, comprising: using the bitter melon plant, or a part thereof, of this invention as a source of said breeding material. Such plant breeding techniques can include, for example, recurrent selection, backcrossing, pedigree breeding, restriction fragment length polymorphism enhanced selection, genetic marker enhanced selection, double haploid breeding, single seed descent, multiple seed descent, and transformation. A bitter melon plant, or a part thereof, produced by this method is also provided herein.

Furthermore, the present invention provides a method for producing a CBM12-derived bitter melon plant, comprising: (a) crossing CBM12, representative seed of said CBM12 having been deposited under ATCC Accession No. ______, with a second bitter melon plant to yield progeny bitter melon seed; (b) growing said progeny bitter melon seed, under plant growth conditions, to yield said CBM12-derived bitter melon plant; (c) crossing said CBM12-derived bitter melon plant with itself or another bitter melon plant to yield additional CBM12-derived progeny bitter melon seed; (d) growing said progeny bitter melon seed of step (a) under plant growth conditions, to yield additional CBM12-derived bitter melon plants; and (e) repeating the crossing and growing steps of (a) and (b) from 0 to 7 times to generate further CBM12-derived bitter melon plants.

Further embodiments of this invention include a method of introducing a desired trait into CBM12, comprising: (a) crossing CBM12, representative seed of said CBM12 having been deposited under ATCC Accession No. ______, with a second bitter melon plant that comprises a desired trait to produce F₁ progeny; (b) selecting an F₁ progeny that comprises the desired trait; (c) crossing the selected F₁ progeny with CBM12, representative seed of said CBM12 having been deposited under ATCC Accession No. ______, to produce backcross progeny; (d) selecting backcross progeny comprising the desired trait and the physiological and morphological characteristics of CBM12; and (e) repeating steps (c) and (d) from 0 to 7 times in succession to produce selected backcross progeny that comprise the desired trait and the physiological and morphological characteristics of CBM12 when grown in the same environmental conditions. In such a method, the trait can be, for example, herbicide tolerance, insect resistance, disease resistance, nematode resistance, tolerance to abiotic stress, yield enhancement, improved fruit characteristics, altered reproductive capability, and altered chemical composition. The present invention also provides a bitter melon plant produced by this method.

In yet further aspects of this invention, a bitter melon product produced from the bitter melon plant of this invention is provided.

These and other aspects of the invention are set forth in more detail in the description of the invention below.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, nucleotide sequences, amino acid sequences, accession numbers and other references cited herein are incorporated by reference in their entireties for the teachings relevant to the sentence and/or paragraph in which the reference is presented.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of a compound (e.g., an amount of a phytomedicine) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

As used herein, the term “plant” includes plant cells, plant protoplasts and plant tissue (e.g., in culture; tissue culture) from which plants can be regenerated, plant calli, plant clumps, and plant cells that are intact in plants or parts of plants, such as leaves, pollen, embryos, cotyledon, hypocotyl, roots, root tips, anthers, flowers and a part thereof, ovules, shoots, stems, stalks, pith, fruit (e.g., gourd, pepo), and the like.

As used herein, the term “tissue culture” encompasses cultures of plant tissue, cells, protoplasts and callus. Methods of culturing plant tissue, cells, protoplasts and callus, as well as methods of regenerating plants from plant tissue cultures are well known in the art.

As used herein, the term “resistance” and the term “tolerance” refer to the ability of a plant to withstand exposure to an insect, a disease or pathogen, an herbicide or other agent or condition (abiotic or biotic). A resistant or tolerant plant variety will have a level of resistance or tolerance, respectively, which is higher than a comparable wild-type variety.

The term “transgene” as used herein, refers to any nucleic acid sequence used in the transformation of a plant or other organism. Thus, a transgene can be a coding sequence, a non-coding sequence, a cDNA, a gene or fragment or portion thereof, a genomic sequence, a regulatory element and the like.

The term “crop” as used herein refers to cultivated plants or agricultural produce, such as grain, vegetables, or fruit, considered as a group.

### Description of the Variety

A breeder uses various methods to help determine which plants should be selected from segregating populations and ultimately which inbred lines will be used to develop hybrids for commercialization. In addition to knowledge of the germplasm and plant genetics, a part of the selection process is dependent on experimental design coupled with the use of statistical analysis. Experimental design and statistical analysis are used to help determine which plants, which family of plants, and finally which inbred lines and hybrid combinations are significantly better or different for one or more traits of interest.

### Breeding History

### Pedigree and Breeding Methods Employed

The medicinal variety, CBM12, was developed by employing a ‘single-seed-descent’ strategy from a wildly grown roadside plant belonging to Momordica charantia L. var. muricata, an allied botanical variety of Momordica charantia var. charantia to which most of the popularly grown horticultural varieties belong. This was one of the two original plants collected at Greenville, S.C. in 2007 and named as accession CBM1 and CBM2. The CBM2 plant was immediately planted in a greenhouse at Clemson University. Female flowers of this plant were selfed to produce selfed fruits. First generation selfed seeds (S₁) were collected from 15 selfed fruits. Fifteen seeds, one each from the fruits were planted in a greenhouse. The 15 plants originated from selfing were named CBM2-S₁-1 through CBM2-S₁-15. This single-seed-descent procedure was repeated until the sixth selfing generation that resulted in 15 S₆ seed-lots originated from CBM2, named CBM2-S₆-1 through CBM2-S₆-15 in summer 2008.

Fifteen S₆ plants were grown using one seed from each of the S₆ seed-lots. The best S₆ plants from the pedigree of CBM2 was selected based on the content of cucurbitacin-B and charantin, these having anticancer and antidiabetic properties, respectively, in its fruits and the selected accession named CBM2-S₆-12. Homozygosity in this selected S₆ progeny was confirmed by AFLP (amplified fragment length polymorphism) marker analysis of the DNA of seedlings produced from 24 randomly selected seeds from the S₆ seed-lots. This selected genotype was CBM12 for brevity. This genotype was compared to 18 other cultivars of diverse geographical origin (six countries) and three genotypes developed in Clemson University (CBM9, CBM10 and CBM18) grown under controlled conditions in a greenhouse during fall 2008 with regard to their fruit weight and content of cucurbitacin-B and charantin. Later, this genotype was formally considered for release as a medicinal variety by the Crop Variety Recommendation and Release Committee of Clemson University in March 2010 and will hereinafter be described as a variety.

The method of selfing that was the same in all six generations; conditions of growing the plants; extraction and quantification of the two phytomedicines; and DNA-fingerprinting followed for development and selection of the variety are discussed below.

### Methods of Selfing

Bitter melon is a monoecious and highly cross-pollinated species and therefore controlled selfing was practiced in each of the six selfing generations. For selfing, a female flower was bagged with a butter paper bag in the afternoon one day prior to blooming (opening of the floral-bud). An intact male flower opened on the day of selfing was plucked and its anthers were rubbed on the stigma of the female flower, followed by immediate re-bagging and labeling. The bags were taken out five days after selfing, assuring formation of the fruit (conversion of ovary to fruit). All the seeds from the ripened fruits were collected, cleaned and sun-dried for raising plants for the succeeding generation.

The seed-to-seed life cycle of the plants was short requiring only 3.5 months and plants could be grown throughout the year under controlled greenhouse conditions. This facilitated development of the variety within less than two years.

### Growing Conditions

The plants were grown in the greenhouse of Clemson University (latitude 34° 41′0″ N, longitude 82° 50′15″ W) at 80° C. (day) and 65° C. (night) temperature and relative humidity of 70-80% (dehumidification set at 80%). Seeds were scarified using sand paper and soaked in sterile distilled water for germination. Germinated seeds were planted in 3×6 (10″×20″) plastic trays (1020 tray and 1801 insert, Landmark Plastic Corp.) and kept under mist in a greenhouse for two days and then transferred to the main greenhouse and kept in the trays until the seedlings attained a height of about 6″ with two completely expanded primary leaves. Thereafter the seedlings were transplanted in 3-gallon pots (10.5″ diameter, 9.5″ height) filled with Fafard 3B Mix. Pots were watered once a day. Peter Excel fertilizer solution of 15:5:15 of N, P and K, respectively (Scotts Corp.), was prepared by dissolving 3 lbs. of solid fertilizer in 3 gallons of water (ca 120,000 ppm) and was applied to the pots once in a week. Plant protection chemicals were applied as required to control diseases and insect-pests. Each plant was provided about 40″×24″ spacing on greenhouse benches (Ludy Greenhouse MFG Corp.).

### Extraction and Quantification of Phytomedicines

Extraction of cucurbitacin-B and charantin was performed using a Soxhlet extraction protocol from 1 g of powder of freeze-dried fruit chips. A final precipitate was dissolved in 200 μl of 1:1 (v/v) chloroform-methanol mixture, and then adjusted to 2 ml with methanol. High-performance liquid chromatography (HPLC) analysis for cucurbitacin-B and charantin was performed with a ZorBAX Eclipse XDB-Phenyl column (5 μm particle, 4.6×250 mm ID) in a mobile phase of 100:2 (v/v) methanol-water. Peaks of the germplasm samples corresponding to the standard peaks from pure samples of cucurbitacin-B and charantin were used for identification and quantification at wavelengths of 235 nm and 205 nm, respectively.

### DNA-Fingerprinting

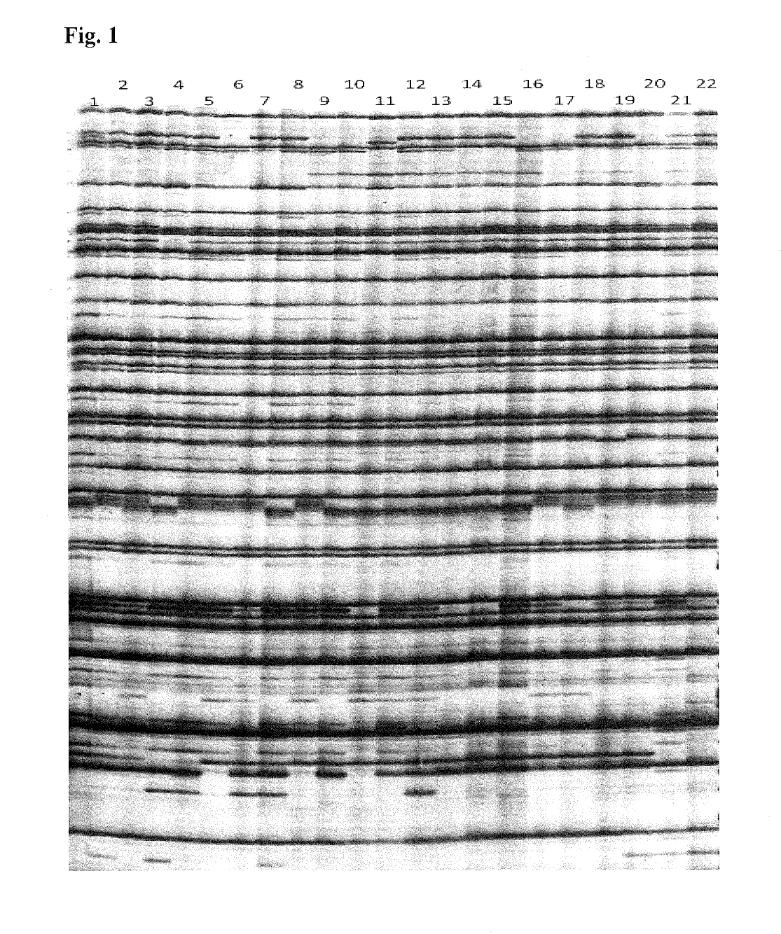

Homozygosity of the variety was verified by DNA-fingerprinting of 24 randomly selected S₆ seedlings employing amplified fragment length polymorphism (AFLP) markers (Vos et al. 1995). AFLP marker analysis was also done on all of the 22 accessions including CBM12 to develop DNA-fingerprints of the variety. AFLP DNA-fingerprinting for this purpose was performed using 10 primer combinations that produced 255 polymorphic marker loci after digesting with the restriction enzymes EcoR1 and Mse1. FIG. 1 depicts the DNA-fingerprint of CBM12 vis-a-vis 21 other genotypes.

### Morphological Features

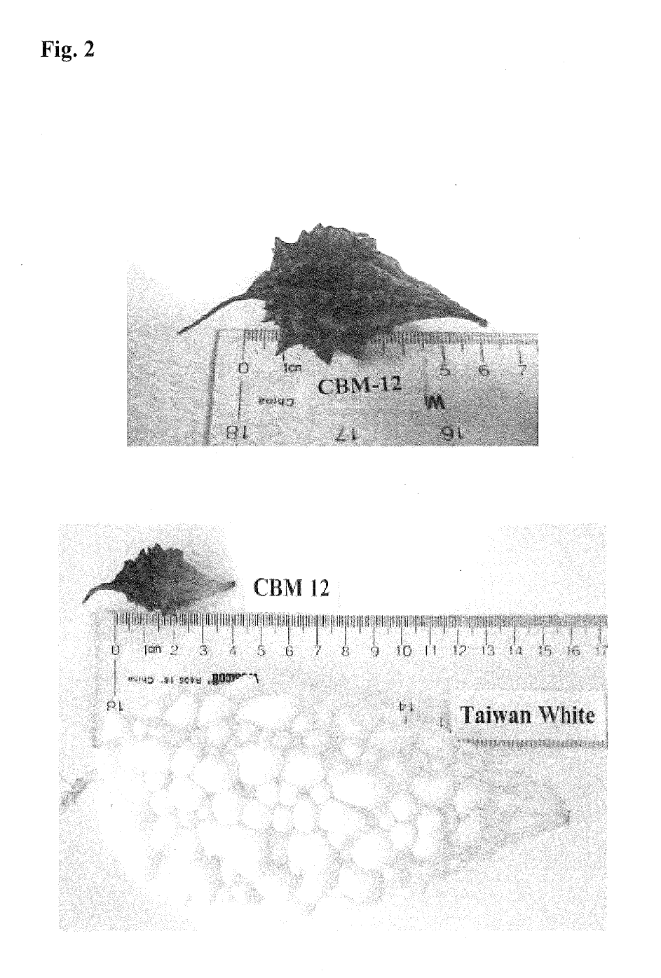

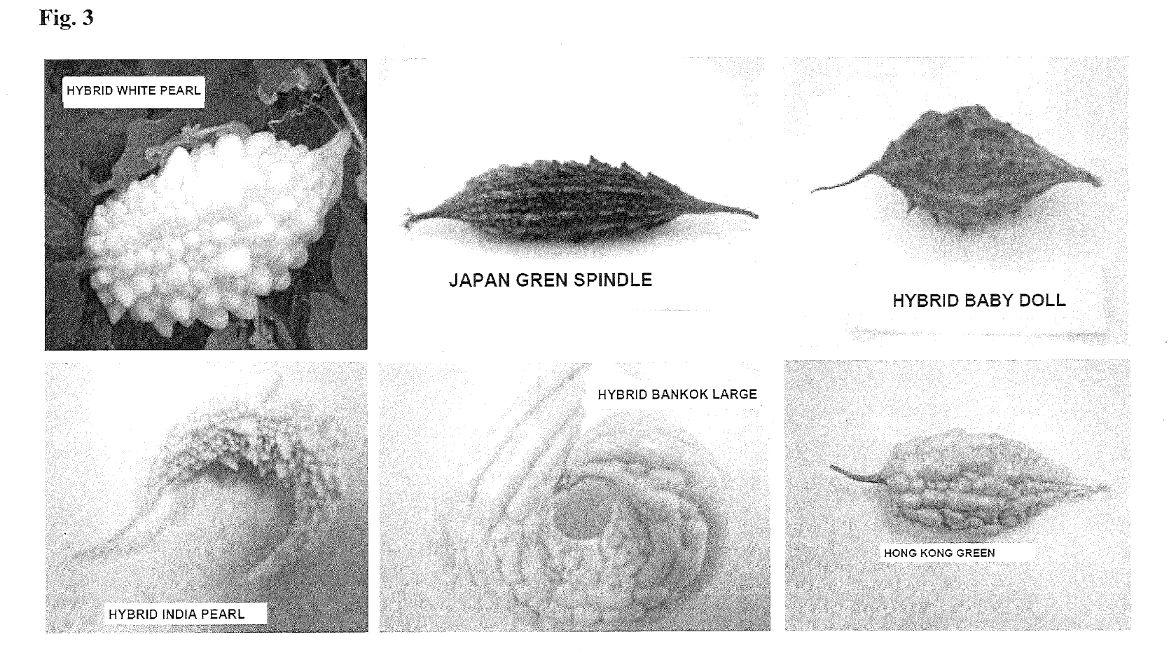

The morphological features of CBM12 in comparison to a popular cultivated variety, Taiwan White, are provided in Table 1. FIGS. 2 and 3 depict fruit attributes (size, color, luster, shape and surface texture) of this variety and its comparison to those of seven randomly selected varieties including Taiwan white.

### Comparative Performance

### Phytomedicine Contents

Contents of the anticancer phytomedicine, cucurbitacin-B (CCR-B), and the antidiabetes phytomedicine, charantin (CHR), of CBM12 along with 16 cultivated varieties and one advanced breeding line (CBM18) are furnished in Table 2. The variation in the contents of CCR-B and CHR among the 19 genotypes was estimated by the Duncan's multiple range test and found to be highly significant at p-value of <0.0001. The average CCR-B and CHR content among the genotypes ranged from 0.3 to 1.0 mg/g and 0.65 to 1.35 mg/g of lyophilized fruit powders, respectively

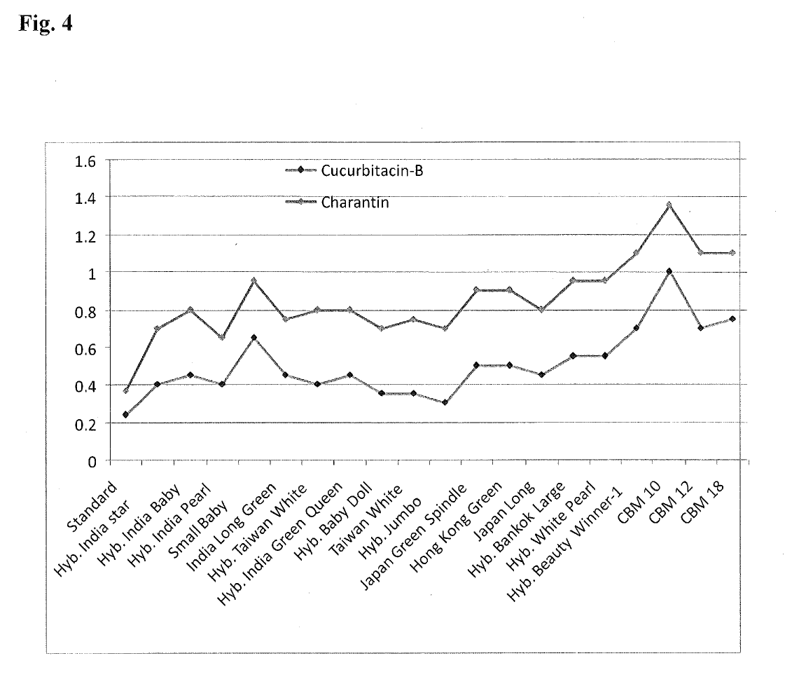

In general, the variety CBM12 along with CBM10, CBM18 and Hybrid Beauty Winner-1 exhibited significantly higher contents of CCR-B and CHR as compared to all other cultivars (FIG. 4; Table 2). Following means comparison using Duncan's multiple range test, CBM10 was found to have significantly the highest contents for CCR-B and CHR as compared with any other genotype. The mean of CHR content for CBM12, CBM18 and Hybrid Beauty Winner-1 was not significantly different, while the mean content of CCR-B was significantly higher in CBM18 as compared to CBM12 and Hybrid Beauty Winner-1, which were not significantly different (Table 2). The variety Hybrid Beauty Winner-1 is supplied by the private company, Asian Vegetable Seeds-Evergreen Seeds (www.evergreenseeds.com/bitgourbitme.html) and this variety, similarly with 17 other commercial varieties used in these studies is not known to be registered or patented. CBM12 has been used to cross with Taiwan White to develop an F₂ segregating population to map the genes controlling some fruit traits and will be used to map the genes controlling the content of some phytomedicines.

### Fruit Weight

CBM12 was proposed and recommended to be used as a medicinal variety for basic and applied research applications in the disciplines of genetics, genomics, physiology, biochemistry, pharmacognosy, microbiology, food science and human nutrition, etc. This genotype can be immediately useful as a donor parent for developing dual-purpose varieties in bitter melon. Fruit of this variety can be used also for consumption since small amounts of fruits or fruit products will be enough to provide high amounts of the anticancer and antidiabetes phytomedicines. The fruit weight of CBM12 was compared with 18 cultivated varieties, three genotypes, CBM9, CBM10 and CBM18, growing under randomized block design (RBD) with three replications in greenhouse. Analysis of Variance (ANOVA) revealed highly significant variation among these 22 genotypes, evidenced by a very high F-value (128.58) and a very high P-value (<0.0001). CBM12 showed (along with CBM10, CBM18, Hybrid India Baby, Small Baby and Hybrid Baby Doll) the lowest fruit weight after the Duncan's multiple range test for means comparison (Table 2).

### Other Representative Embodiments of the Invention

The present invention also encompasses hybrid plants produced from bitter melon variety CBM12, plants derived from CBM12, and CBM12 plants comprising a nucleic acid that has been introduced therein by traditional breeding or genetic engineering techniques, and seeds, plant parts, and tissue cultures of the foregoing plants, as well as methods of producing the plants of the invention.

Accordingly, methods for crossing the bitter melon plants of the present invention are provided. Such methods may comprise crossing the plant of the present invention, CBM12, with itself or a second bitter melon plant. The present invention further encompasses a method for producing hybrid bitter melon seed, the method comprising crossing two bitter melon plants and harvesting the resultant hybrid bitter melon seed, wherein at least one bitter melon plant is the bitter melon plant of the present invention, CBM12. In another embodiment, a method for producing a first generation (F₁) hybrid bitter melon seed is provided comprising crossing the plant of the present invention with a different bitter melon plant and harvesting the resultant first generation (F₁) hybrid bitter melon seed. Further provided by the present invention are plants produced by these methods.

Additionally provided herein is a method for producing a CBM12-derived bitter melon plant comprising: (a) crossing bitter melon variety CBM12 with a second bitter melon plant to yield progeny bitter melon seed; (b) growing said progeny bitter melon seed, under plant growth conditions, to yield said or CBM12-derived bitter melon plant. The method may still further comprise: a) crossing said CBM12-derived bitter melon plant with itself or another bitter melon plant to yield additional CBM12-derived progeny bitter melon seed; (b) growing said progeny bitter melon seed of step (a) under plant growth conditions, to yield additional CBM12-derived bitter melon plants; and (c) repeating the crossing and growing steps of (a) and (b) multiple times to generate further CBM12-derived bitter melon plants. In some embodiments, the crossing and growing steps of (a) and (b) in step (c) are repeated from 0 to 2 times, from 0 to 3 times, from 0 to 4 times, 0 to 5 times, from 0 to 6 times, from 0 to 7 times, from 0 to 8 times, from 0 to 9 times or from 0 to 10 times, in order to generate further CBM12-derived bitter melon plants. In other embodiments, the crossing and growing steps of (a) and (b) in step (c) are repeated from 0 to n times in order to generate further CBM12-derived bitter melon plants. The invention further provides plants produced by these methods. Accordingly, the invention encompasses progeny plants and parts thereof with at least one ancestor that is hybrid bitter melon plant CBM12 and more specifically where the pedigree of this progeny includes 1, 2, 3, 4, 5, 6, and/or 7 cross pollinations to a bitter melon plant CBM12 or a plant that has CBM12 as a progenitor.

Other embodiments of the present invention include a method for producing a bitter melon plant that contains in its genetic material one or more transgenes, comprising crossing the bitter melon plant of the present invention with either a second plant of another bitter melon line, or a non-transformed bitter melon plant of the present invention, wherein progeny are produced, so that the genetic material of the progeny that result from the cross comprises the transgene(s) operably linked to one or more regulatory elements. In one aspect of the invention, the one or more transgene includes but is not limited to a nucleic acid conferring herbicide resistance, insect resistance, disease resistance and/or altered reproductive capability. Also provided by the present invention are plants produced by this method.

Further provided by the present invention is a method for developing a bitter melon plant in a bitter melon plant breeding program using plant breeding techniques, which include employing a bitter melon plant of the present invention, or a part thereof, as the source of plant breeding material. Plant breeding techniques that can be used in the method include, but are not limited to, recurrent selection, backcrossing, pedigree breeding, restriction fragment length polymorphism enhanced selection, genetic marker enhanced selection, double haploid breeding, single seed descent, multiple seed descent, and/or transformation. Further provided herein are plants produced by this method.

Accordingly, any methods using the variety CBM12 are part of this invention: selfing, backcrosses, hybrid production, crosses to populations, and the like. All plants produced using CBM12 as a parent are within the scope of this invention including plants derived from CBM12. Advantageously CBM12 cultures used in crosses with other bitter melon plants can be used to produce a first generation (F₁) bitter melon hybrid seed and plants with superior characteristics.

Other aspects of the present invention include products produced from the variety CBM12 and methods for producing those products.

Accordingly, one aspect of the present invention is a bitter melon product produced from the bitter melon plant designated CBM12, representative seed of said bitter melon variety CBM12 having been deposited under ATCC Accession No. ______. Thus, products of the present invention comprise products produced from any part of the bitter melon plant. Including but not limited to, fruits, seeds, leaves, pollen, embryos, cotyledons, hypocotyls, roots, root tips, anthers, flowers, ovules, shoots, stems, stalks, pith, fruit (e.g., gourd, pepo), wood, and the like.

In addition to the above discussed bitter melon products and methods of making or producing such bitter melon products, the bitter melon variety CBM12 is also useful for producing other commercially valuable products including, but not limited to, food additives, human food products and livestock food products.

Accordingly, in some embodiments, the present invention provides a product produced from the bitter melon plant designated CBM12, representative seed of said bitter melon variety CBM12 having been deposited under ATCC Accession No. ______, wherein the product produced is selected from the group consisting of food additives, human food products and animal food products.

In other aspects of the present invention a method of sustaining a human or animal subject is provided, the method comprising feeding the subject the bitter melon of the bitter melon variety CBM12. The bitter melon of the present invention may be fed as a food product comprising the bitter melon of the present invention alone or in combination with at least one additional nutrient. Thus, a food product as used herein can be used for humans and/or animals.

Examples of subjects of this invention can include, but are not limited to, humans, non-human primates, dogs, cats, horses, cattle, goats, pigs, sheep, guinea pigs, mice, rats and rabbits, as well as any other domestic, commercially or clinically valuable animal, including animal models and livestock animals.

Human subjects can be male and/or female and can be of any age including neonates, infants, juveniles, adults and/or senescent individuals.

A food product of the present invention can comprise the bitter melon plant CBM12. The plant can be transgenic, carrying one or more heterologous genes of interest, or it can be non-transgenic. The plant and/or fruit of the plant can be included in the food product in any suitable form as would be known in the art. The food product may comprise any amount of the plant and/or fruit (or plant or fruit part), for example from 1, 2, 3, 4, 5 or 10 percent to 90, 95 or 99 percent by weight (or more), with the balance of the product comprising other nutrients or additives.

Other nutrients or additives that can be combined with plants or plant parts as described herein to produce a food product of the invention can include carbohydrates fats, lipids, and/or proteins. Carbohydrate sources used to produce a food product according to the present invention include, but are not limited to, corn, oats, barley, sorghum, or combinations thereof, which can be ground into a meal for use in the food. Supplementary protein sources include, but are not limited to, Fraction I and Fraction II proteins, soy meal, fish meal, blood meal, poultry by-product (ground poultry offal), meat meal, feather-lysate (see, e.g., U.S. Pat. No. 4,908,220 to Shih) and combinations thereof. The plant or plant parts can be the sole protein source, or supplemented as described above. Other nutrients in small amounts, such as vitamins, minerals, antibiotics, and other substances or compounds can be included in the food. The ingredients may be mixed and blended in accordance with any procedure known in the art of food products formulated into any suitable form, including, but not limited to, food pellets, beverages and food bars. The bitter melon of the present invention can be genetically modified to provide for the production of additional nutrients therein, if desired.

### Hybrid Production

The development of bitter melon hybrids involves, in general, the development of completely homozygous lines, the crossing of these lines, and the evaluation of the crosses. In the case of bitter melon, a completely homozygous line may be an inbred or a doubled-haploid line.

Pedigree breeding and recurrent selection breeding methods are typically used to develop inbred lines from breeding populations. Breeding programs combine the genetic backgrounds from two or more inbred lines or various other germplasm sources into breeding pools from which new inbred lines are developed by selfing and selection of desired phenotypes. The new inbreds are crossed with other inbred lines or doubled-haploid lines, and the hybrids from these crosses are evaluated to determine which have commercial potential.

Pedigree breeding starts with the crossing of two genotypes, each of which may have one or more desirable characteristics lacking in the other or which complements the other. If the two original parents do not provide all the desired characteristics, other sources can be included in the breeding population. In the pedigree method, superior plants are selfed and selected in successive generations. In the succeeding generations, the heterozygous condition gives way to homogeneous lines as a result of self-pollination and selection. Typically in the pedigree method of breeding, five or more generations of selfing and selection is practiced. Thus, multiple crossings and growing steps may be carried out in order to generate a desired hybrid.

A single cross bitter melon hybrid results from the cross of two bitter melon lines (e.g., inbred or doubled-haploid lines), each of the parents having a genotype that complements the genotype of the other. The hybrid progeny of the first generation is designated F₁. Preferred F1 hybrids may be more vigorous than either parent in a cross between inbred parents. This hybrid vigor, or heterosis, can be manifested in many polygenic traits, including increased vegetative growth and increased yield.

In general, the development of a bitter melon hybrid involves three steps: (1) the selection of plants from various germplasm pools for initial breeding crosses; (2) the selfing of the selected plants from the breeding crosses for several generations to produce a series of inbred lines, which, although different from each other, breed true and are highly uniform; and (3) crossing the selected inbred lines with different inbred lines to produce the hybrid progeny (F₁). A consequence of the homozygosity and homogeneity of the inbred lines is that the hybrid between a defined pair of inbreds/doubled-haploids will always be the same. Once the parents that give a superior hybrid have been identified, the hybrid seed can be reproduced indefinitely as long as the homogeneity of the parents is maintained.

A single cross hybrid is produced when two lines are crossed to produce the F₁ progeny. A double cross hybrid is produced from four inbred lines crossed in pairs (A×B and C×D) and then the two F₁ hybrids are crossed again (A×B)×(C×D). Much of the hybrid vigor exhibited by F₁ hybrids is generally lost in the next generation (F₂). Consequently, seed from hybrids is not typically used for planting stock.

### Evaluation of Plants for Homozygosity and Phenotypic Stability

It is desirable and advantageous for a bitter melon variety to be highly homogeneous, homozygous and phenotypically uniform and stable for use as a commercial variety or breeding material. In the case of inbreds or other lines, there are many analytical methods available to determine the homozygotic and phenotypic stability of the variety.

The oldest and most traditional method of analysis is the observation of phenotypic traits. The data are usually collected in field experiments over the life of the bitter melon plants to be examined. Phenotypic characteristics most often observed are for traits associated with seed yield, disease resistance, maturity, plant height, internode distance, flower color, leaf color, leaf yield, leaf size, leaf angle, lamina-midrib ratio, and concentration of chemical components such as phytomedicines.

In addition to phenotypic observations, the genotype of a plant can also be examined. There are many laboratory-based techniques available for the analysis, comparison and characterization of plant genotypes; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Simple Sequence Repeats (SSRs), which are also referred to as Microsatellites.

The presence or absence of a marker in the plant genotype may be determined by any method known in the art. For example, the marker sequence (or its complement) may be used as a hybridization probe, e.g., for Southern or in situ analysis of genomic DNA. Typically, however, due to greater ease and sensitivity, an amplification method, such as PCR will be used to detect the presence or absence of the marker in the plant genotype.

Molecular markers can be used in any method of nucleic acid amplification known in the art. Such methods include but are not limited to Polymerase Chain Reaction (PCR; described in U.S. Pat. Nos. 4,683,195; 4,683,202; 4,800,159; 4,965,188), Strand Displacement Amplification (SDA; described by G. Walker et al., Proc. Nat. Acad. Sci. USA 89: 392 (1992); G. Walker et al., Nucl. Acids Res. 20: 1691 (1992); U.S. Pat. No. 5,270,184), thermophilic Strand Displacement Amplification (tSDA; EP 0 684 315 to Frasier et al.), Self-Sustained Sequence Replication (3SR; J. C. Guatelli et al., Proc Natl. Acad. Sci. USA 87: 1874-78 (1990)), Nucleic Acid Sequence-Based Amplification (NASBA; U.S. Pat. No. 5,130,238 to Cangene), the Qβ replicase system (P. Lizardi et al., BioTechnology 6: 1197 (1988)), or transcription based amplification (D. Y. Kwoh et al., Proc. Natl. Acad. Sci. USA 86: 1173-77 (1989)).

Transfer of Traits into Bitter Melon Cultivar CBM12

Genetic variants of CBM12 that are naturally-occurring or created through traditional breeding methods using variety CBM12 are also intended to be within the scope of this invention. In particular embodiments, the invention encompasses plants of variety CBM12 and parts thereof further comprising one or more additional traits, in particular, specific, single gene transferred traits. Examples of traits that may be transferred include, but are not limited to, herbicide resistance, disease resistance (e.g., bacterial fungal or viral disease), nematode resistance, tolerance to abiotic stresses (e.g., drought, temperature, salinity), yield enhancement, improved nutritional quality (e.g., oil starch and protein content or quality), altered chemical composition (e.g., phytomedicines), improved fruit characteristics (color, shape, size, number, chemical composition), altered reproductive capability (e.g., male sterility) or other agronomically important traits.

Such traits may be introgressed into variety CBM12 from another bitter melon variety or may be directly transformed into variety CBM12 (discussed below). One or more new traits can be transferred to variety CBM12, or, alternatively, one or more traits of variety CBM12 are altered or substituted. The introgression of the trait(s) into variety CBM12 may be achieved by any method of plant breeding known in the art, for example, pedigree breeding, backcrossing, doubled-haploid breeding, and the like (see, Wernsman, E. A., and Rufty, R. C. 1987. Chapter Seventeen. Bitter melon. Pages 669-698 In: Cultivar Development. Crop Species. W. H. Fehr (ed.), MacMillan Publishing Co., Inc., New York, N.Y. 761 pp.).

The laboratory-based techniques described above, in particular RFLP and SSR, can be used in such backcrosses to identify the progenies having the highest degree of genetic identity with the recurrent parent. This permits one to accelerate the production of bitter melon varieties having at least 90%, at least 95%, at least 99% or greater genetic identity with the recurrent parent and further comprising the trait(s) introgressed from the donor parent. Such determination of genetic identity can be based on molecular markers used in the laboratory-based techniques described herein.

The last backcross generation can be selfed to give pure breeding progeny for the nucleic acid(s) being transferred. The resulting plants generally have essentially all of the morphological and physiological characteristics of variety CBM12, in addition to the transferred trait(s) (e.g., one or more single gene traits). The exact backcrossing protocol will depend on the trait being altered to determine an appropriate testing protocol. Although backcrossing methods are simplified when the trait being transferred is a dominant allele, a recessive allele may also be transferred. In this instance, it may be necessary to introduce a test of the progeny to determine if the desired trait has been successfully transferred.

Those skilled in the art will appreciate that the bitter melon nucleic acids described below in connection with bitter melon plants produced by genetic engineering techniques may also be introduced into the variety CBM12 by conventional breeding methods.

### Transformation of Bitter Melon

With the advent of molecular biological techniques that have allowed the isolation and characterization of nucleic acids that encode specific protein products, scientists in the field of plant biology developed a strong interest in engineering the genome of plants to contain and express foreign nucleic acids, or additional, or modified versions of native or endogenous nucleic acids (perhaps driven by different promoters) in order to alter the traits of a plant in a specific manner. Such foreign, additional and/or modified nucleic acids are referred to herein collectively as “transgenes.” The term “transgene” as used herein, refers to any nucleic acid sequence used in the transformation of a plant or other organism. Thus, a transgene can be a coding sequence, a non-coding sequence, a cDNA, a gene or fragment or portion thereof, a genomic sequence, a regulatory element and the like. Thus, the term “transgene,” as used herein, is not necessarily intended to indicate that the foreign nucleic acid is from a different plant species. For example, the transgene may be a particular allele derived from another bitter melon line or may be an additional copy of an endogenous gene. Several methods for producing transgenic plants are known in the art. Therefore, in particular embodiments, the present invention also encompasses transformed versions of the bitter melon variety CBM12.

Plant transformation generally involves the construction of an expression vector that will function in plant cells. Such a vector comprises DNA or RNA comprising a nucleic acid under control of, or operatively linked to, a regulatory element (for example, a promoter). The expression vector may contain one or more such operably linked nucleic acid/regulatory element combinations. The vector(s) may be in the form of, for example, a plasmid or a virus, and can be used, alone or in combination with other vectors, to provide transformed bitter melon plants, using transformation methods as described below to incorporate transgenes into the genetic material of the bitter melon plant(s).

Any transgene(s) known in the art may be introduced into a bitter melon plant, tissue, cell or protoplast according to the present invention, e.g., to improve commercial or agronomic traits, herbicide resistance, disease resistance (e.g., to a bacterial fungal or viral disease), insect resistance, nematode resistance, yield enhancement, nutritional quality (e.g., oil starch and protein content or quality), fruit characteristics (color, shape, size, number, chemical composition), and/or altered reproductive capability and/or chemical composition (e.g., phytomedicines). Alternatively, a transgene may be introduced for the production of recombinant proteins (e.g., enzymes) or metabolites.

In particular embodiments of the invention, a transgene conferring herbicide resistance, insect resistance, or disease resistance is introduced into the bitter melon plant.

### Expression Vectors For Bitter Melon Transformation

### Genetic Markers

Expression vectors typically include at least one genetic marker, operably linked to a regulatory element (a promoter, for example) that allows transformed cells containing the marker to be either recovered by negative selection, i.e., inhibiting growth of cells that do not contain the selectable marker, or by positive selection, i.e., screening for the product encoded by the genetic marker. Many commonly used selectable marker for plant transformation are well known in the transformation art, and include, for example, nucleic acids that code for enzymes that metabolically detoxify a selective chemical agent which may be an antibiotic or a herbicide, or nucleic acids that encode an altered target which is insensitive to the inhibitor. Positive selection methods are also known in the art.

One commonly used selectable marker for plant transformation is neomycin phosphotransferase II (npfII), isolated from transposon Tn5, which when placed under the control of plant regulatory signals confers resistance to kanamycin (Fraley et al., (1983) Proc. Natl. Acad. Sci. U.S.A. 80: 4803). Another commonly used selectable marker is hygromycin phosphotransferase, which confers resistance to the antibiotic hygromycin (Vanden Elzen et al., (1985) Plant Mol. Biol. 5: 299).

Additional selectable markers of bacterial origin that confer resistance to antibiotics include gentamycin acetyl transferase, streptomycin phosphotransferase, aminoglycoside-3′-adenyl transferase and the bleomycin resistance determinant (Hayford et al., (1988) Plant Physiol. 86: 1216; Jones et al., (1987) Mol. Gen. Genet., 210: 86; Svab et al., (1990) Plant Mol. Biol. 14: 197; Hille et al., (1986) Plant Mol. Biol. 7: 175). Other selectable markers confer resistance to herbicides, such as glyphosate, glufosinate and bromoxynil (Comai et al., (1985) Nature 317: 741; Gordon-Kamm et al., (1990) Plant Cell 2: 603; and Stalker et al., (1988) Science 242: 419).

Selectable markers for plant transformation that are not of bacterial origin include, for example, mouse dihydrofolate reductase, plant 5-eno/pyruvylshikimate-3-phosphate synthase and plant acetolactate synthase (Eichholtz et al., (1987) Somatic Cell Mol. Genet. 13: 67; Shah et al., (1986) Science 233: 478; Charest et al., (1990) Plant Cell Rep. 8: 643).

Another class of markers for plant transformation requires screening of presumptively transformed plant cells rather than direct genetic selection of transformed cells for resistance to a toxic substance such as an antibiotic. These markers are particularly useful to quantify or visualize the spatial pattern of expression in specific tissues and are frequently referred to as reporters because they can be fused to a nucleic acid or regulatory sequence for the investigation of nucleic acid expression. Commonly used reporters for screening presumptively transformed cells include β□-glucuronidase (GUS), β□-galactosidase, luciferase and chloramphenicol acetyltransferase (Jefferson, R. A., (1987) Plant Mot. Biol. Rep. 5: 387; Teeri et al., (1989) EMBO J. 8: 343; Koncz et al., (1987) Proc. Natl. Acad. Sci. U.S.A. 84:131; De Block et al., (1984) EMBO J. 3: 1681).

In vivo methods for visualizing GUS activity that do not require destruction of plant tissue are also available (Molecular Probes Publication 2908, Imagene Green™, p. 1-4 (1993) and Naleway et al., (1991) J. Cell Biol. 115: 15). However, these in vivo methods for visualizing GUS activity have not proven useful for recovery of transformed cells because of low sensitivity, high fluorescent backgrounds, and limitations associated with the use of luciferase as a selectable marker.

In addition, a nucleic acid encoding green fluorescent protein (GFP) has been utilized as a marker for nucleic acid expression in prokaryotic and eukaryotic cells (Chalfie et al., (1994) Science 263: 802). GFP and mutants of GFP may be used as screenable markers.

### Promoters

Nucleic acids included in expression vectors are typically driven by a nucleotide sequence comprising a regulatory element, for example, a promoter. Several types of promoters are now well known in the transformation art, as are other regulatory elements that can be used alone or in combination with promoters.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences. A “plant promoter” is a promoter capable of initiating transcription in plant cells. Such promoters include those that drive expression of a nucleotide sequence constitutively, those that drive expression when induced, and those that drive expression in a tissue- or developmentally specific manner, as these various types of promoters are known in the art.

### Constitutive Promoters

Thus, for example, in some embodiments of the invention, a constitutive promoter can be used to drive the expression of a transgene in a plant cell. A constitutive promoter is an unregulated promoter that allows for continual transcription of its associated coding sequence. Thus, constitutive promoters are generally active under most environmental conditions, in most or all cell types and in most or all states of development or cell differentiation.

Any constitutive promoter functional in a plant can be utilized in the instant invention. Exemplary constitutive promoters include, but are not limited to, the promoters from plant viruses including, but not limited to, the 35S promoter from CaMV (Odell et al., Nature 313: 810 (1985)); figwort mosaic virus (FMV) 35S promoter (P-FMV35S, U.S. Pat. Nos. 6,051,753 and 6,018,100); the enhanced CaMV35S promoter (e35S); the 1′- or 2′-promoter derived from T-DNA of Agrobacterium tumefaciens; the nopaline synthase (NOS) and/or octopine synthase (OCS) promoters, which are carried on tumor-inducing plasmids of Agrobacterium tumefaciens (Ebert et al., Proc. Natl. Acad. Sci. USA, 84:5745 5749, 1987); actin promoters including, but not limited to, rice actin (McElroy et al., Plant Cell 2: 163 (1990); U.S. Pat. No. 5,641,876); histone promoters; tubulin promoters; ubiquitin and polyubiquitin promoters ((Sun and Callis, Plant J., 11(5):1017-1027 (1997)); Christensen et al., Plant Mol. Biol. 12: 619 (1989) and Christensen et al., Plant Mol. Biol. 18: 675 (1992)); pEMU (Last et al., Theor. Appl. Genet. 81: 581 (1991)); the mannopine synthase promoter (MAS) (Velten et al., EMBO J. 3: 2723 (1984)); maize H3 histone (Lepelit et al., Mol. Gen. Genet. 231: 276 (1992) and Atanassova et al., Plant Journal 2: 291 (1992)); the ALS promoter, a XbaI/NcoI fragment 5′ to the Brassica napus ALS3 structural gene (or a nucleotide sequence that has substantial sequence similarity to said XbaI/NcoI fragment); ACT11 from Arabidopsis (Huang et al., Plant Mol. Biol. 33:125-139 (1996)); Cat3 from Arabidopsis (GenBank® Database Accession No. U43147, Zhong et al., Mol. Gen. Genet. 251:196-203 (1996)); GPc1 from maize (GenBank® Database Accession No. X15596, Martinez et al., J. Mol. 208:551-565 (1989)); and Gpc2 from maize (GenBank® Database Accession No. U45855, Manjunath et al., Plant Mol. Biol. 33:97-112 (1997)).

### Inducible Promoters

In some embodiments of the present invention, an inducible promoter can be used to drive the expression of a transgene. Inducible promoters activate or initiate expression only after exposure to, or contact with, an inducing agent. Inducing agents include, but are not limited to, various environmental conditions (e.g., pH, temperature), proteins and chemicals. Examples of environmental conditions that can affect transcription by inducible promoters include pathogen attack, anaerobic conditions, extreme temperature and/or the presence of light. Examples of chemical inducing agents include, but are not limited to, herbicides, antibiotics, ethanol, plant hormones and steroids. Any inducible promoter that is functional in a plant can be used in the instant invention (see, Ward et al., (1993) Plant Mol. Biol. 22: 361 (1993)). Exemplary inducible promoters include, but are not limited to, that from the ACEI system, which responds to copper (Melt et al., PNAS 90: 4567 (1993)); the In2 nucleic acid from maize, which responds to benzenesulfonamide herbicide safeners (Hershey et al., (1991) Mol. Gen. Genetics 227: 229 (1991) and Gatz et al., Mol. Gen. Genetics 243: 32 (1994)); a heat shock promoter, including, but not limited to, the soybean heat shock promoters Gmhsp 17.5-E, Gmhsp 17.2-E and Gmhsp 17.6-L and those described in U.S. Pat. No. 5,447,858; the Tet repressor from Tn10 (Gatz et al., Mol. Gen. Genet. 227: 229 (1991)) and the light-inducible promoter from the small subunit of ribulose bisphosphate carboxylase (ssRUBISCO). Other examples of inducible promoters include, but are not limited to, those described by Moore et al. (Plant J. 45:651-683 (2006)). Additionally, some inducible promoters respond to an inducing agent to which plants do not normally respond. An example of such an inducible promoter is the inducible promoter from a steroid hormone gene, the transcriptional activity of which is induced by a glucocorticosteroid hormone (Schena et al., Proc. Natl. Acad. Sci. USA 88: 421 (1991)).

### Tissue-Specific or Tissue-Preferred Promoters

In further embodiments of the present invention, a tissue-specific promoter can be used to drive the expression of a transgene in a particular tissue in the transgenic plant. Tissue-specific promoters drive expression of a nucleic acid only in certain tissues or cell types, e.g., in the case of plants, in the leaves, stems, flowers and their various parts, roots, fruits and/or seeds, etc. Thus, plants transformed with a nucleic acid of interest operably linked to a tissue-specific promoter produce the product encoded by the transgene exclusively, or preferentially, in a specific tissue or cell type.

Any plant tissue-specific promoter can be utilized in the instant invention. Exemplary tissue-specific promoters include, but are not limited to, a root-specific promoter, such as that from the phaseolin gene (Murai et al., (1983) Science 23: 476 and Sengupta-Gopalan et al., (1985) Proc. Natl. Acad. Sci. USA 82: 3320); a leaf-specific and light-induced promoter such as that from cab or rubisco (Simpson et al. (1985) EMBO J. 4: 2723 and Timko et al., (1985) Nature 318: 579); the fruit-specific E8 promoter from tomato (Lincoln et al. Proc. Nat'l. Acad. Sci. USA 84: 2793-2797 (19.88); Deikman et al. EMBO J. 7: 3315-3320 (1988); Deikman et al. Plant Physiol. 100: 2013-2017 (1992); seed-specific promoters of, for example, Arabidopsis thaliana (Krebbers et al. (1988) Plant Physiol. 87:859); an anther-specific promoter such as that from LAT52 (Twell et al. (1989) Mol. Gen. Genet. 217: 240) or European Patent Application No 344029, and those described by Xu et al. (Plant Cell Rep. 25:231-240 (2006)) and Gomez et al. (Planta 219:967-981 (2004)); a pollen-specific promoter such as that from Zm13 (Guerrero et al., (1993) Mol. Gen. Genet. 224: 161), and those described by Yamaji et al. (Plant Cell Rep. 25:749-57 (2006)) and Okada et al. (Plant Cell Physiol. 46:749-802 (2005)); a pith-specific promoter, such as the promoter isolated from a plant TrpA gene as described in International PCT Publication No. WO93/07278; and a microspore-specific promoter such as that from apg (Twell et al. (1993) Sex. Plant Reprod. 6: 217). Exemplary green tissue-specific promoters include the maize phosphoenol pyruvate carboxylase (PEPC) promoter, small subunit ribulose bis-carboxylase promoters (ssRUBISCO) and the chlorophyll a/b binding protein promoters.

### Signal Sequences For Targeting Proteins to Subcellular Compartments

Transport of proteins produced by transgenes to a subcellular compartment such as the chloroplast, vacuole, peroxisome, glyoxysome, cell wall or mitochondrion, or for secretion into the apoplast, may be accomplished by means of operably linking a nucleotide sequence encoding a signal sequence typically at the 5′ and/or 3′ region of a sequence encoding the protein of interest. Association of targeting sequences with the coding sequence may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art (see, for example, Becker et al., (1992) Plant Mol. Biol. 20: 49; Close, P. S., Master's Thesis, Iowa State University (1993); Knox, C., et al., (1987) Plant Mol. Biol. 9: 3; Lerner et al., (1989) Plant Physiol. 91: 124; Fontes et al., (1991) Plant Cell 3: 483; Matsuoka et al., (1991) Proc. Natl. Acad. Sci. 88: 834; Gould et al., (1989) J. Cell Biol 108: 1657; Creissen et al., (1991) Plant J. 2: 129; Kalderon et al., (1984) Cell 39: 499; Stiefel et al., (1990) Plant Cell 2: 785).

Foreign Nucleic Acids that May be Introduced into Bitter Melon Plants

Nucleic acids of agronomic importance can be expressed in transformed plants. More particularly, plants can be genetically engineered to express various phenotypes of agronomic interest. Exemplary nucleic acids implicated in this regard include, but are not limited to, those described below.

As an example, a transgene whose expression results in or contributes to a desired trait to be transferred to variety CBM12 can comprise a nucleic acid encoding an insecticidal protein, such as, for example, a crystal protein of Bacillus thuringiensis or a vegetative insecticidal protein from Bacillus cereus, such as VIP3 (see, for example, Estruch et al. (1997) Nat Biotechnol 15:137).

In a further embodiment, a transgene introduced into variety CBM12 can comprise a nucleic acid conferring herbicide tolerance, the expression of which renders plants of variety CBM12 tolerant to the herbicide. For example, expression of an altered acetohydroxyacid synthase (AHAS) enzyme confers upon plants tolerance to various imidazolinone or sulfonamide herbicides (U.S. Pat. No. 4,761,373). In a still further embodiment, a nucleic acid conferring tolerance to imidazolinones or sulfonylurea herbicides is transferred into variety CBM12. Expression of a mutant acetolactate synthase (ALS) will render the plants resistant to inhibition by sulfonylurea herbicides (U.S. Pat. No. 5,013,659).

U.S. Pat. No. 4,975,374 describes plant cells and plants containing a nucleic acid encoding a mutant glutamine synthetase (GS) which confers resistance to herbicides that are known to inhibit GS, e.g., phosphinothricin and methionine sulfoximine. In addition, expression of a Streptomyces bar nucleic acid encoding a phosphinothricin acetyl transferase results in tolerance to the herbicide phosphinothricin or glufosinate (U.S. Pat. No. 5,489,520). U.S. Pat. No. 5,162,602 discloses plants tolerant to inhibition by cyclohexanedione and aryloxyphenoxypropanoic acid herbicides. The tolerance is conferred by an altered acetyl coenzyme A carboxylase (ACCase). U.S. Pat. No. 5,554,798 discloses transgenic glyphosate tolerant plants, which tolerance is conferred by an altered 5-enolpyruvyl-3-phosphoshikimate (EPSP) synthase nucleic acid. In another particular embodiment, tolerance to a protoporphyrinogen oxidase inhibitor is achieved by expression of a tolerant protoporphyrinogen oxidase enzyme in plants (U.S. Pat. No. 5,767,373). In another particular embodiment, a nucleic acid transferred into variety CBM12 can comprise a transgene conferring tolerance to a herbicide and at least one other transgene conferring another trait, such as for example, insect resistance or tolerance to another herbicide.

For purposes of proprietary protection of a transgenic plant, a genetic map can be generated, for example, via conventional Restriction Fragment Length Polymorphism (RFLP) analysis, Polymerase Chain Reaction (PCR) analysis, and/or Simple Sequence Repeats (SSR), which identifies the approximate chromosomal location of the integrated DNA molecule. For exemplary methodologies in this regard, see Glick and Thompson, METHODS IN PLANT MOLECULAR BIOLOGY AND BIOTECHNOLOGY 269-284 (CRC Press, Boca Raton, 1993). Thus, if unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and/or sequencing, all of which are conventional techniques.

### Transgenes That Confer Resistance To Pests or Disease

(A) Plant disease resistance. Plant defenses are often activated by specific interaction between the product of a nucleic acid coding for disease resistance (R) in the plant and the product of a corresponding nucleic acid coding for avirulence (Avr) in the pathogen. A plant variety can be transformed with a cloned nucleic acid conferring resistance in order to engineer plants that are resistant to specific pathogens (see, for example, Jones et al., (1994) Science 266: 789, cloning of the tomato Cf-9 gene for resistance to Cladosporium fulvum; Martin et al., (1993) Science 262: 1432, tomato Pto gene for resistance to Pseudomonas syringae pv.; Mindrinos et al., (1994) Cell 78: 1089, Arabidopsis RSP2 nucleic acid encoding resistance to Pseudomonas syringae).

(B) A Bacillus thuringiensis protein, a derivative thereof or a synthetic polypeptide modeled thereon (see, for example, Geiser et al., (1986) Gene 48: 109, disclosing the cloning and nucleotide sequence of Bt δ-endotoxin) can be introduced via a transgene. Moreover, nucleic acid molecules encoding δ-endotoxincan be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and patent applications and hereby are incorporated by reference for this purpose: U.S. Pat. Nos. 5,188,960; 5,689,052; 5,880,275; WO 91/14778; WO 99/31248; WO 01/12731; WO 99/24581; WO 97/40162 and U.S. application Ser. Nos. 10/032,717; 10/414,637; and 10/606,320.

(C) A lectin (see, for example, the disclosure by Van Damme et al., (1994) Plant Molec. Biol. 24: 25, which discloses the nucleotide sequences of several Clivia miniata mannose-binding lectins) can be introduced via a transgene.

(D) A vitamin-binding protein such as avidin (see PCT publication WO 93/06487, which teaches the use of avidin and avidin homologues as larvicides against insect pests) can be introduced via a transgene.

(E) An enzyme inhibitor, for example, a protease inhibitor or an amylase inhibitor (see, for example, Abe et al., (1987) J. Biol. Chem. 262: 16793, nucleotide sequence of rice cysteine proteinase inhibitor; Huub et al., (1993) Plant Molec. Biol. 21: 985; nucleotide sequence of cDNA encoding bitter melon proteinase inhibitor 1; and Sumitani et al., (1993) Biosci. Biotech. Biochem. 57: 1243, nucleotide sequence of Streptomyces nitrosporeus amylase inhibitor) can be introduced via a transgene.

(F) An insect-specific hormone or pheromone such as an ecdysteroid or juvenile hormone, a variant thereof, a mimetic based thereon, or an antagonist or agonist thereof (see, for example, the disclosure of Hammock et al., (1990) Nature 344: 458, of baculovirus expression of cloned juvenile hormone esterase, an inactivator of juvenile hormone) can be introduced via a transgene.

(G) An insect-specific peptide or neuropeptide which, upon expression, disrupts the physiology of the affected pest can be introduced via a transgene (for example, see the disclosures of Regan, (1994) J. Biol. Chem. 269: 9, expression cloning yields DNA coding for insect diuretic hormone receptor; Pratt et al., (1989) Biochem. Biophys. Res. Comm. 163: 1243, an allostatin is identified in Diploptera puntata; Chattopadhyay et al. (2004) Crit. Rev. Microbiol. 30 (1): 33 54 2004; Zjawiony (2004) J. Nat. Prod. 67 (2): 300 310; Carlini & Grossi-de-Sa (2002) Toxicon, 40 (11): 1515 1539; Ussuf et al. (2001) Curr. Sci. 80 (7): 847 853; and Vasconcelos & Oliveira (2004) Toxicon 44 (4): 385-403. See also U.S. Pat. No. 5,266,317 to Tomalski et al., which discloses nucleic acids encoding insect-specific, paralytic neurotoxins.

(H) An insect-specific venom produced in nature by a snake, a wasp, or the like can be introduced via a transgene (see, e.g., Pang et al., (1992) Gene 116: 165, for disclosure of heterologous expression in plants of a nucleic acid encoding a scorpion insectotoxic peptide).

(I) An enzyme responsible for an hyperaccumulation of a monterpene, a sesquiterpene, a steroid, hydroxamic acid, a phenylpropanoid derivative or another non-protein molecule with insecticidal activity can be introduced via a transgene.

(J) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and/or a glucanase, whether natural or synthetic, can be introduced via a transgene (see, for example, PCT application WO 93/02197 in the name of Scott et al., which discloses the nucleotide sequence of a callase). DNA molecules which contain chitinase-encoding sequences can be obtained, for example, from the ATCC under Accession Nos. 39637 and 67152 (see also Kramer et al., (1993) Insect Biochem. Molec. Biol. 23: 691, which describes the nucleotide sequence of a cDNA encoding tobacco hookworm chitinase, and Kawalleck et al., (1993) Plant Molec. Biol. 21: 673, which provides the nucleotide sequence of parsley ubi4-2 polyubiquitin).

(K) A molecule that stimulates signal transduction can be introduced via a transgene. For example, see the disclosure by Botella et al., (1994) Plant Molec. Biol. 24: 757, of nucleotide sequences for mung bean calmodulin cDNA clones, and Griess et al., (1994) Plant Physiol. 104: 1467, which provides the nucleotide sequence of a maize calmodulin cDNA clone.

(L) A hydrophobic moment peptide can be introduced via a transgene (see PCT application WO 95/16776 which discloses peptide derivatives of Tachyplesin which inhibit fungal plant pathogens, and PCT application WO 95/18855 which teaches synthetic antimicrobial peptides that confer disease resistance).

(M) A membrane permease, a channel former or a channel blocker can be introduced via a transgene. For example, see the disclosure by Jaynes et al., (1993) Plant Sci. 89: 43), of heterologous expression of a cecropin-β□ lytic peptide analog to render transgenic plants resistant to Pseudomonas solanacearum.

(N) A viral-invasive protein or a complex toxin derived therefrom can be introduced via a transgene. For example, the accumulation of viral coat proteins in transformed plant cells imparts resistance to viral infection and/or disease development effected by the virus from which the nucleic acid encoding the coat protein is derived, as well as by related viruses (see Beachy et al., (1990) Ann. Rev. Phytopathol. 28: 451). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus (Id.).

(O) An insect-specific antibody or an immunotoxin derived therefrom can be introduced via a transgene. Thus, an antibody targeted to a critical metabolic function in the insect gut would inactivate an affected enzyme, killing the insect (Cf. Taylor et al., Abstract #497, SEVENTH INT'L SYMPOSIUM ON MOLECULAR PLANT-MICROBE INTERACTIONS (Edinburgh, Scotland, 1994); enzymatic inactivation in transgenic tobacco via production of single-chain antibody fragments).

(P) A virus-specific antibody can be introduced via a transgene (see, for example, Taviadoraki et al., (1993) Nature 366: 469; showing that transgenic plants expressing recombinant antibody are protected from virus attack).

(Q) A developmental-arrestive protein produced in nature by a pathogen or a parasite can be introduced via a transgene. Thus, fungal endo α-1,4-D-polygalacturonases facilitate fungal colonization and plant nutrient release by solubilizing plant cell wall homo-□α-1,4-D-galacturonase (see Lamb et al., (1992) Bio/Technology 10: 1436). The cloning and characterization of a nucleic acid which encodes a bean endopolygalacturonase-inhibiting protein is described by Toubart et al., (1992) Plant J. 2: 367.

(R) A developmental-arrestive protein produced in nature by a plant can be introduced via a transgene. For example, Logemann et al., (1992) Bio/Technology 10: 305, shows that transgenic plants expressing the barley ribosome-inactivating nucleic acid have an increased resistance to fungal disease.

(S) Nucleic acids involved in the Systemic Acquired Resistance (SAR)

Response and/or the pathogenesis related nucleic acids can be introduced via a transgene. Briggs, S., Current Biology, 5(2) (1995), Pieterse & Van Loon (2004) Curr. Opin. Plant Bio. 7(4):456 64 and Somssich (2003) Cell 113(7):815 6.

(T) Nucleic acids encoding resistance to fungi can be introduced via a transgene (Cornelissen and Melchers, Pl. Physiol. 101:709 712, (1993) and Parijs et al., Planta 183:258 264, (1991) and Bushnell et al., Can. J. Plant Pathol. 20(2):137 149 (1998). Also see U.S. application Ser. No. 09/950,933.

### Transgenes That Confer Resistance To A Herbicide

(A) An herbicide that inhibits the growing point or meristem, such as an imidazalinone or a sulfonylurea can be introduced via a transgene. Exemplary transgenes or nucleic acids in this category code for mutant ALS or AHAS enzyme as described, for example, by Lee et al., (1988) EMBO J. 7: 1241, and Miki et al., (1990) Theor. Appl. Genet. 80: 449, respectively.

(B) Glyphosate (resistance imparted by mutant 5-enolpyruvl-3-phosphikimate synthase (EPSP) and aroA nucleic acids) and other phosphono compounds such as glufosinate (phosphinothricin acetyl transferase (PAT) and Streptomyces hygroscopicus phosphinothricin acetyl transferase (bar) nucleic acids), and pyridinoxy or phenoxy proprionic acids and cycloshexones (ACCase inhibitor-encoding nucleic acids) can be introduced via a transgene. See, for example, U.S. Pat. No. 4,940,835 to Shah et al., which discloses the nucleotide sequence of a form of EPSP which can confer glyphosate resistance. A DNA molecule encoding a mutant aroA can be obtained under ATCC Accession No. 39256, and the mutant nucleotide sequence is disclosed in U.S. Pat. No. 4,769,061 to Comai. European Patent Application No. 0 333 033 to Kumada et al. and U.S. Pat. No. 4,975,374 to Goodman et al. disclose nucleotide sequences encoding glutamine synthetase, which confers resistance to herbicides such as L-phosphinothricin. The nucleotide sequence encoding a phosphinothricin-acetyl-transferase is provided in European Application No. 0 242 246 to Leemans et al. De Greef et al., (1989) Bio/Technology 7: 61, describes the production of transgenic plants that express chimeric bar coding for phosphinothricin acetyl transferase activity. Exemplary nucleic acids conferring resistance to phenoxy proprionic acids and cycloshexones, such as sethoxydim and haloxyfop, are the Acct-S1, Acc1-S2 and Acc1-S3 nucleic acids described by Marshall et al., (1992) Theor. Appl. Genet. 83: 435.

(C) An herbicide that inhibits photosynthesis, such as a triazine (psbA and gs+) and/or a benzonitrile (nitrilase) can be introduced via a transgene. Przibilla et al., (1991) Plant Cell 3: 169, describe the transformation of Chlamydomonas with plasmids encoding mutant psbA. Nucleic acids encoding nitrilase are disclosed in U.S. Pat. No. 4,810,648 to Stalker, and these nucleic acids are available under ATCC Accession Nos. 53435, 67441 and 67442. Cloning and expression of DNA coding for a glutathione S-transferase is described by Hayes et al., (1992) Biochem. J. 285: 173.

### Transgenes That Confer Or Contribute To A Value-Added Trait

(A) Decreased phytate content: Introduction of a phytase-encoding nucleic acid would enhance breakdown of phytate, adding more free phosphate to the transformed plant. For example, see Van Hartingsveldt et al., (1993) Gene 127: 87, for a disclosure of the nucleotide sequence of an Aspergillus niger phytase.

(B) Modified carbohydrate composition effected, for example, by transforming plants with a nucleic acid encoding an enzyme that alters the branching pattern of starch (see Shiroza et al., (1998) J. Bacteriol. 170: 810, nucleotide sequence of Streptococcus mutans fructosyltransferase; Steinmetz et al., (1985) Mol. Gen. Genet. 200: 220, nucleotide sequence of Bacillus subtilis levansucrase; Pen et al., (1992) Bio/Technology 10: 292, production of transgenic plants that express Bacillus licheniformis α□-amylase; Elliot et al., (1993) Plant Molec. Biol. 21: 515, nucleotide sequences of tomato invertase; Søgaard et al., (1993) J. Biol. Chem. 268: 22480, site-directed mutagenesis of barley α-amylase nucleic acid; and Fisher et al., (1993) Plant Physiol. 102: 1045, maize endosperm starch branching enzyme II).

Those skilled in the art will appreciate that the transgenes described above may also be transferred into bitter melon plants using conventional breeding techniques as known in the art and as described herein.

As a further alternative, the transgene can encode an antisense nucleic acid molecule or any other non-translated nucleic acid as known in the art. In a further alternative embodiment, the transgene effects gene suppression in the host plant.

### Methods for Bitter Melon Transformation

Plants can be transformed according to the present invention using any suitable method known in the art. Intact plants, plant tissue, explants, meristematic tissue, protoplasts, callus tissue, cultured cells, and the like may be used for transformation depending on the plant species and the method employed. Procedures for transforming a wide variety of plant species are well known and routine in the art and described throughout the literature. Such methods include, but are not limited to, transformation via bacterial-mediated nucleic acid delivery, viral-mediated nucleic acid delivery, silicon carbide or nucleic acid whisker-mediated nucleic acid delivery, liposome mediated nucleic acid delivery, microinjection, microparticle bombardment, electroporation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Bacterial mediated nucleic acid delivery includes but is not limited to DNA delivery by Agrobacterium spp. and is described, for example, in Horsch et al. (Science 227:1229 (1985); Ishida et al. (Nature Biotechnol. 14:745 750 (1996); and Fraley et al. (Proc. Natl. Acad. Sci. 80: 4803 (1983)). Transformation by various other bacterial species is described, for example, in Broothaerts et al. (Nature 433:629-633 (2005)).

Physical delivery of nucleotide sequences via microparticle bombardment is also well known and is described, for example, in Sanford et al. (Methods in Enzymology 217:483-509 (1993)) and McCabe et al. (Plant Cell Tiss. Org. Cult. 33:227-236 (1993)).

Another method for physical delivery of nucleic acid to plants is sonication of target cells. This method is described, for example, in Zhang et al. (Bio/Technology 9:996 (1991)). Nanoparticle-mediated transformation is another method for delivery of nucleic acids into plant cells (Radu et al., J. Am. Chem. Soc. 126: 13216-13217 (2004); Torney, et al. Society for In Vitro Biology, Minneapolis, Minn. (2006)). Alternatively, liposome or spheroplast fusion can be used to introduce nucleotide sequences into plants. Examples of the use of liposome or spheroplast fusion are provided, for example, in Deshayes et al. (EMBO J., 4:2731 (1985), and Christou et al. (Proc Natl. Acad. Sci. U.S.A. 84:3962 (1987)). Direct uptake of nucleic acid into protoplasts using CaCl₂ precipitation, polyvinyl alcohol or poly-L-ornithine is described, for example, in Hain et al. (Mol. Gen. Genet. 199:161 (1985)) and Draper et al. (Plant Cell Physiol. 23:451 (1982)). Electroporation of protoplasts and whole cells and tissues is described, for example, in Donn et al. (In Abstracts of VIIth International Congress on Plant Cell and Tissue Culture IAPTC, A2-38, p 53 (1990); D'Halluin et al. (Plant Cell 4:1495-1505 (1992)); Spencer et al. (Plant Mol. Biol. 24:51-61 (1994)) and Fromm et al. (Proc. Natl. Acad. Sci. 82: 5824 (1985)). Polyethylene glycol (PEG) precipitation is described, for example, in Paszkowski et al. (EMBO J. 3:2717 2722 (1984)). Microinjection of plant cell protoplasts or embryogenic callus is described, for example, in Crossway (Mol. Gen. Genetics 202:179-185 (1985)). Silicon carbide whisker methodology is described, for example, in Dunwell et al. (Methods Mol. Biol. 111:375-382 (1999)); Frame et al. (Plant J. 6:941-948 (1994)); and Kaeppler et al. (Plant Cell Rep. 9:415-418 (1990)).

Plant cells, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. 1,1984, and Vol. II, 1986). It is known that practically all plants can be regenerated from cultured cells or tissues.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture medium will generally contain various amino acids and plant hormones, such as auxin and cytokinins. A large number of plants have been shown capable of regeneration from transformed individual cells to obtain transgenic whole plants.

The regenerated plants are transferred to standard soil conditions and cultivated in a conventional manner. The plants are grown and harvested using conventional procedures.

The foregoing methods for transformation may be used for producing transgenic inbred lines. Transgenic inbred lines can then be crossed, with another (non-transformed or transformed) inbred line, in order to produce a transgenic hybrid bitter melon plant. Alternatively, a genetic trait that has been engineered into a particular bitter melon line using the foregoing transformation techniques can be moved into another line using traditional backcrossing techniques that are well known in the plant breeding arts. For example, a backcrossing approach can be used to move an engineered trait from a non-elite line into an elite bitter melon line, or from a hybrid bitter melon plant containing a foreign nucleic acid in its genome into a line or lines, which do not contain that nucleic acid. As used above, “crossing” can refer to a simple X by Y cross, or the process of backcrossing, depending on the context.

### Products

Bitter melon plants, or parts thereof, of the present invention may be utilized in any product containing bitter melon and/or bitter melon fruit, including, but not limited to, shredded bitter melon, cut bitter melon, ground bitter melon, granulated bitter melon, fine particulate bitter melon, powder bitter melon fruit, dried bitter melon fruit (e.g., freeze dried, sun dried), bitter melon fruit chips and/or bitter melon extract. Accordingly, some embodiments of the invention provide bitter melon products produced from the plants of the present invention, or parts thereof. In further embodiments of the present invention, the bitter melon product may contain other bitter melon varieties or bitter melon types. In still other embodiments, the bitter melon products of the invention may contain any other bitter melon type or bitter melon product constituents in any form. Additionally, the bitter melon products of the present invention can include a flavoring component or scent, as described below.

The bitter melon plants of the present invention, or parts thereof, can be used to produce bitter melon products wherein the bitter melon products have increased levels of the phytomedicines cucurbitacin-B and charantin. The bitter melon plants of the invention, or parts thereof, can be used in blends with bitter melon from other bitter melon varieties to make a blended bitter melon product. Thus, the bitter melon plants of the invention, or parts thereof, can be used to produce blended bitter melon products with increased levels of cucurbitacin-B and charantin. The bitter melon plants of the present invention can also be used for human and/or animal food products.

Plant parts that can be collected from the plants of the present invention (e.g., cut or harvested) include, but are not limited to, fruits, seeds, leaves, pollen, embryos, cotyledons, hypocotyls, roots, root tips, anthers, flowers, ovules, shoots, stems, stalks, pith, fruit (e.g., gourd, pepo), wood, etc.

A bitter melon formulation for a bitter melon product can incorporate other components in addition to bitter melon which can alter the bitterness, sweetness, sourness or saltiness of the formulation; enhance the perceived dryness or moistness of the formulation; or the degree of bitter melon taste exhibited by the formulation. Such other components may be salts (e.g., sodium chloride, potassium chloride, sodium citrate, potassium citrate, sodium acetate, potassium acetate, and the like); natural sweeteners (e.g., fructose, sucrose, glucose, maltose, mannose, galactose, lactose, and the like); artificial sweeteners (e.g., sucralose, saccharin, aspartame, acesulfame K, and the like), organic and inorganic fillers (e.g., grains, processed grains, puffed grains, maltodextrin, dextrose, calcium carbonate, calcium phosphate, corn starch, lactose, manitol, xylitol, sorbitol, finely divided cellulose, and the like); binders (e.g., povidone, sodium carboxymethylcellulose and other modified cellulosic types of binders, sodium alginate, xanthan gum, starch-based binders, gum arabic, lecithin, and the like); pH adjusters or buffering agents (e.g., metal hydroxides, preferably alkali metal hydroxides such as sodium hydroxide and potassium hydroxide, and other alkali metal buffers such as potassium carbonate, sodium carbonate, sodium bicarbonate, and the like); colorants (e.g., dyes and pigments, including caramel coloring and titanium dioxide, and the like); humectants (e.g. glycerin, propylene glycol, and the like); preservatives (e.g., potassium sorbate, and the like); syrups (e.g., honey, high fructose corn syrup, and the like); disintegration aids (e.g., microcrystalline cellulose, croscarmellose sodium, crospovidone, sodium starch glycolate, pregelatinized corn starch, and the like); antioxidants (e.g., ascorbic acid, grape seed extracts and oils, polyphenol-containing materials such as green tea extract and black tea extract, peanut endocarb, potato peel, and the like (See Santhosh et al., Phytomedicine, 122:16-220 (2005); incorporated herein by reference); and flavoring agents. Flavoring agents may be natural or synthetic, and include, but are not limited to, fresh, sweet, herbal, confectionary, floral, fruity or spice. Specific types of flavors include, but are not limited to, vanilla, coffee, chocolate, cream, mint, spearmint, menthol, peppermint, wintergreen, lavender, cardamon, nutmeg, cinnamon, clove, cascarilla, sandalwood, honey, jasmine, ginger, anise, sage, licorice, lemon, orange, apple, peach, lime, cherry, and strawberry. Flavorings also can include components that are considered moistening, cooling or smoothening agents, including, but not limited to, eucalyptus. These flavors may be provided alone or in a composite (e.g., spearmint and menthol, or orange and cinnamon). Representative types of components are also set forth in U.S. Pat. No. 5,387,416 to White et al. and PCT Application Publication No. WO 2005/041699 to Quinter et al., the relevant portions of each of which is incorporated herein by reference.

### INDUSTRIAL APPLICABILITY

This invention is also directed to methods for producing a bitter melon plant by crossing a first parent bitter melon plant with a second parent bitter melon plant wherein either the first or second parent bitter melon plant is a bitter melon plant of variety CBM12 or a bitter melon plant of variety CBM12 further comprising one or more additional traits (e.g., single gene traits). Further, both first and second parent bitter melon plants can come from CBM12 or a bitter melon plant of variety CBM12 further comprising one or more traits (e.g., single gene traits). Thus, any such methods using the bitter melon variety CBM12 or a bitter melon plant of CBM12 further comprising one or more additional traits (e.g., one or more single gene traits) are part of this invention: selfing, backcrosses, doubled-haploid production, hybrid production, crosses to populations, and the like. All plants produced using bitter melon variety CBM12 or modified variety CBM12 further comprising one or more additional traits (e.g., one or more single gene traits) as a parent are within the scope of this invention.

Advantageously, bitter melon variety CBM12 or modified variety CBM12 further comprising one or more additional traits (e.g., one or more single gene traits) can be used in crosses with other, different, bitter melon inbreds to produce first generation (F₁) bitter melon hybrid seeds and plants with superior characteristics.

### Deposits

A deposit of at least ______ seeds of bitter melon variety CBM12 has been made with the American Type Culture Collection (ATCC), Manassas, Va. 20110 USA on ______. The deposit has been assigned ATCC Accession Number ______. This deposit of the bitter melon variety CBM12 will be maintained in the ATCC depository, which is a public depository, for a period of 30 years, or 5 years after the most recent request, or for the effective life of the patent, whichever is longer, and will be replaced if it becomes nonviable during that period. Applicants do not waive any infringement of their rights granted under this patent or under the Plant Variety Protection Act (7 U.S.C. 2321 et seq.).

TABLE 1
Objective description of the varieties.

| | | CBM12 | Taiwan White |
|---|---|---|---|
| Species | | charantia | charantia |
| Subspecies | | muricata | charantia |
| Kind | | Gourd | Gourd |
| Type | | Summer (vegetable marrow) | Summer (vegetable marrow) |
| Mature plant | Growth habit | Long vines | Long vines |
| | Plant type | Pilose | Pilose |
| Main stem | Section shape | Angular | Angular |
| | Diameter (mm)- mid-point of 1st internode | 14 | 30 |
| | Average length (cm) | Indeterminate | Indeterminate |
| | Average number of nodes | Indeterminate | Indeterminate |
| Leaves | Blade shape | Ovate | Ovate |
| | Blade form | Deep lobed | Deep lobed |
| | Margin | Spiny | Spiny |
| | Margin edges | Flat | Flat |
| | Width (cm) | 7 | 9.5 |
| | Length (cm) | 6 | 9.5 |
| | Leaf surface | Smooth | Smooth |
| | Dorsal surface pubescence | Soft hairy | Soft hairy |
| | Ventral surface pubescence | Soft hairy | Soft hairy |
| | Leaf color | Light green | Light green |
| | Leaf blotching | Not blotched | Not blotched |
| | Petiole length (cm) | 2 | 3.5 |
| Flower- | Diameter (cm) | 2 | 4 |
| Pistillate | Ovary | Fusiform | Fusiform |
| | Pedicel length (cm) | 5.5 | 6 |
| | Margin shape | Curved | Curved |
| | Margin edges | Plain | Plain |
| | Sepal width (mm) | 1 | 2 |
| | Sepal length (mm) | 2.5 | 3 |
| | Color | Lemon yellow | Lemon yellow |
| Flower- | Sepal length (mm) | 6 | 6 |
| Staminate | Sepal width (mm) | 3 | 4 |
| | Pedicel length (cm) | 3.5 | 5.75 |
| | Color | Lemon yellow | Lemon yellow |
| Fruit (Market | Length (cm) | 5.6 | 17.5 |
| maturity) | Width (cm) - widest at middle | 9.8 | 18.6 |
| | Average weight (gm) | 3.8 | 146 |
| | Shape according to variety type | Hubbard | Hubbard |
| | Apex | Taper pointed | Taper pointed |
| | Base | Taper pointed | Taper pointed |
| | Ribs | None | None |
| | Fruit surface | With pointed teeth | With flat teeth |
| | Warts (teeth) | Many | Many |
| | Blossom Scar Button | Slightly extended | Slightly extended |
| Seed cavity | Length (cm) | 1.1 | 1 |
| | Width (cm) | 0.6 | 0.65 |
| | Location | Conforms to fruit shape | Conforms to fruit shape |
| | Placental tissue | Sparse | Sparse |
| | Center core | Prominent | Prominent |
| Fruit stalks | length (cm) | 7 | 6.5 |
| | Diameter (mm) | 1 | 2.5 |
| | Cross-section shape | Round | Round |
| | Twisting | Not twisted | Not twisted |
| | Tapering | Tapered | Tapered |
| | Straightness | Straight | Straight |
| | Texture | Hard | Hard |
| | Furrows | None | None |
| | Surface | Spiny | Rough |
| | Attachment end | Slightly expanded | Slightly expanded |
| | Detaches | With difficulty | With difficulty |
| | Color | Light green | Light green |
| Seeds | Length (mm) | 12 | 12 |
| | Width (mm) | 7 | 8 |
| | Thickness (mm) | 2 | 2.5 |
| | Face surface | Scaly | Scaly |
| | Color | Cream | Black |
| | Luster | Dull | Dull |
| | Margin | Straight | Straight |
| | Margin edge | Rounded | Rounded |
| | Separation from pulp | Moderately easy | Moderately easy |
| | gms per 100 seeds | 11.9 | 19.2 |
| | No. of seeds per fruit | 3 | 14 |
| | Seed coat | Normal | Normal |

TABLE 2
The mean values for fruit weight (FWT), and contents
of cucurbitacin-B (CCR-B) and charantin (CHR) of 22
genotypes, showing statistical significance between varieties
using the Duncan's multiple range test¹.

| Sno | Variety | FWT (g) | CCR-B² | CHR² |
|---|---|---|---|---|
| 1 | Hyb. India Star | 74.83^{bc} | 0.4^{h} | 0.7^{g} |
| 2 | Hyb. India Baby | 4.28^{g} | 0.45^{g} | 0.8^{e} |
| 3 | Hyb. India Pearl | 72.26^{cd} | 0.4^{h} | 0.65^{h} |
| 4 | Small Baby | 3.48^{g} | 0.65^{d} | 0.95^{c} |
| 5 | India Long Green | 22.64^{ef} | 0.45^{g} | 0.75^{f} |
| 6 | Hyb. Taiwan White | 63.6^{d} | 0.4^{h} | 0.8^{e} |
| 7 | Hyb. India Green Queen | 25.29^{e} | 0.45^{g} | 0.8^{e} |
| 8 | Hyb. Baby Doll | 5.1^{g} | 0.35ⁱ | 0.7^{g} |
| 9 | Taiwan White | 146.33^{a} | 0.35ⁱ | 0.75^{f} |
| 10 | Hyb. Jumbo | 70.87^{cd} | 0.3^{j} | 0.7^{g} |
| 11 | Japan Green Spindle | 16.1^{f} | 0.5^{f} | 0.9^{d} |
| 12 | Taiwan Large | 77.63^{bc} | — | — |
| 13 | Hong Kong Green | 70.61^{cd} | 0.5^{f} | 0.9^{d} |
| 14 | Japan Long | 78.69^{bc} | 0.45^{g} | 0.8^{e} |
| 15 | Large Top | 83.87^{b} | — | — |
| 16 | Hyb. Bangkok Large | 78.5b^{c} | 0.55^{e} | 0.95^{c} |
| 17 | Hyb. White Pearl | 82.37^{b} | 0.55^{e} | 0.95^{c} |
| 18 | Hyb. Beauty Winner-1 | 74.81^{bc} | 0.7^{c} | 1.1^{b} |
| 19 | CBM9 | 21.47^{ef} | — | — |
| 20 | CBM10 | 4.9^{g} | 1.0^{a} | 1.35^{a} |
| 21 | CBM12 | 3.84^{g} | 0.7^{c} | 1.1^{b} |
| 22 | CBM18 | 5.89^{g} | 0.75^{b} | 1.1^{b} |

¹No significant difference was observed between the values having the same letter for the Duncan's multiple range tests at 5% probability level.
²mg/g powdered lyophilized fruits

That which is claimed is:

1. A bitter melon seed designated CBM12, representative seed of said CBM12 having been deposited under ATCC Accession No. ______.

2. A bitter melon plant, or a part thereof, produced by the seed of claim 1.

3. Pollen of the plant of claim 2.

4. An ovule of the plant of claim 2.

5. A bitter melon plant, or a part thereof, having all the physiological and morphological characteristics of CBM12, said CBM12 having been deposited under ATCC Accession No. ______.

6. A tissue culture of regenerable cells of the plant, or part thereof, of claim 2.

7. The tissue culture according to claim 6, wherein the regenerable cells are from plant parts selected from the group consisting of leaves, pollen, embryos, cotyledons, hypocotyls, roots, root tips, anthers, flowers and a part thereof, ovules, shoots, stems, stalks, pith and fruit or wherein the regenerable cells are callus or protoplasts derived therefrom.

8. A bitter melon plant regenerated from the tissue culture of claim 6, expressing all the morphological and physiological characteristics of CBM12, said CBM12 having been deposited under ATCC Accession No. ______.

9. A method for producing a first generation (F₁) hybrid bitter melon seed, comprising crossing the plant of claim 2 with a different bitter melon plant and harvesting the resultant first generation (F₁) hybrid bitter melon seed.

10. An F₁ hybrid bitter melon seed produced by the method of claim 9.

11. An F₁ hybrid plant, or a part thereof, grown from the seed of claim 10.

12. A method for producing hybrid bitter melon seed, comprising crossing two bitter melon plants and harvesting the resultant hybrid bitter melon seed, wherein at least one bitter melon plant is the bitter melon plant of claim 2.

13. A method for producing a CBM12-derived bitter melon plant, comprising:
(a) crossing CBM12, representative seed of said CBM12 having been deposited under ATCC Accession No. ______, with a second bitter melon plant to yield progeny bitter melon seed;
(b) growing said progeny bitter melon seed, under plant growth conditions, to yield said CBM12-derived bitter melon plant.

14. A CBM12-derived bitter melon plant, or a part thereof, produced by the method of claim 13.

15. The bitter melon plant, or a part thereof, of claim 2, wherein the plant or a part thereof has been transformed so that its genetic material comprises one or more transgenes operably linked to one or more regulatory elements.

16. A method for producing a bitter melon plant that contains in its genetic material one or more transgenes, comprising crossing the bitter melon plant of claim 15 with either a second plant of another bitter melon line, or a non-transformed bitter melon plant, wherein progeny are produced, so that the genetic material of the progeny that result from the cross comprises the transgene(s) operably linked to one or more regulatory elements.

17. A bitter melon plant, or a part thereof, produced by the method of claim 16.

18. A bitter melon product produced from the bitter melon plant of claim 2.

19. A bitter melon product produced from the bitter melon plant of claim 5.

20. Fruit of the plant of claim 2.
